(19) United States
(12) Patent Application Publication
(10) Pub. No.: US 2024/0024226 A1
(43) Pub. Date: Jan. 25, 2024

(54) EXTRACT OF BACTERIUM OF SPHINGOMONAS GENUS

(71) Applicant: L'OREAL, Paris (FR)

(72) Inventors: Pascal HILAIRE, AULNAY-SOUS-BOIS (FR); Yann MAHE, AULNAY-SOUS-BOIS (FR); Francis PRUCHE, AULNAY-SOUS-BOIS (FR); Gayané AZADIGUIAN, AULNAY-SOUS-BOIS (FR); Muriel BAYER-VANMOEN, CHEVILLY LARUE (FR); Richard MARTIN, ROCHECORBON (FR)

(21) Appl. No.: 18/256,491

(22) PCT Filed: Dec. 17, 2021
(86) PCT No.: PCT/EP2021/086557

§ 371 Date: Jun. 8, 2023

(30) Foreign Application Priority Data
Dec. 18, 2020 (FR) FR2013724

(51) Int. Cl. A61K 8/99; A61K 8/64; A61K 8/9789; A61K 8/19; A61Q 19/00

(52) U.S. Cl. CPC A61K 8/99; A61K 8/64; A61K 8/9789; A61K 8/19; A61Q 19/00

(57) ABSTRACT

The present invention relates to an extract of bacterium of Sphingomonas genus, compositions comprising at least one bacterium of Sphingomonas genus or said extract, the uses thereof, as well as their implementations in cosmetic methods.

The present invention relates to an extract of bacterium of Sphingomonas genus, compositions comprising at least one bacterium of Sphingomonas genus or said extract, the uses thereof, as well as their implementations in cosmetic methods.

Activating the kallikrein/kinin system (KKS) leads to the transformation of the inactive proenzymes factor XII and prekallikrein into enzyme (active form): activated factor XII (FXIIa) and kallikrein (KK). The Kallikrein cleaves the high-molecular weight kininogen (HK) and releases the bradykinin (BK). The bradykinin and its active metabolite desArg9BK bind to the receptors B2 (RB2) and B1 (RB1) respectively. The interaction of the ligands, BK and desArg9BK, with their receptors, RB2 and RB1, leads to vasodilation and vascular vasopermeability resulting in water exiting the vessels to the tissues and possible diapedesis of the cells.

Bradykinine is therefore involved in the vasodilation and the increase in the vascular permeability. Using bradykininergic antagonists is one of the effective alternatives to all cosmetic disorders that make use of the systems described hereinabove.

The present inventors have surprisingly demonstrated that an extract of bacterium of Sphingomonas genus can be used as an inhibitor of the kallikrein-kinin system and therefore attenuate the appearance of redness. Furthermore, they have shown that a composition comprising an extract of Sphingomonas xenophaga on a skin model stimulated by capsaicin makes it possible to significantly decrease the appearance of redness.

Redness of the skin is a skin disorder, mainly present on the face, that can have various origins. It can be of short duration (hot flashes) or can be permanent. Redness of the skin is characterized by capillary vasodilation.

This redness can be triggered by a great variety of factors, such as (i) external stimuli such as the consumption of food or hot or alcoholic beverages, by rapid variations in temperature, by heat or cold, by a low relative humidity, by exposure of the skin to violent winds or drafts (fans, air conditioning), and by the application of certain surfactants, or (ii) redness associated with a skin disorder such as rosacea, acne rosacea, atopic dermatitis, seborrhoeic dermatitis, as well as (iii) redness subsequent to an allergic skin reaction.

These red patches are often unattractive, often uncomfortable and create substantial psychological discomfort.

Skin stimulated by capsaicin is in particular used as a reactive skin model.

Generally, reactive skin is defined by a particular reactivity of the skin.

This skin reactivity generally results in signs of discomfort in response to a contact of the subject with a triggering element that can have various origins. This can be a cold or hot application or the application of certain products on the surface of the reactive skin, exposure to abrupt changes in temperature, etc. There are also associated factors such as the age and the type of skin.

The appearance of these signs of discomfort, which appear within the minutes following contact with the triggering elements, is one of the essential characteristics of the reactive skin. These signs of discomfort are mainly dysesthetic sensations, i.e. more or less unpleasant sensations felt on a skin zone, such as tautness, tingling, itching, heating, discomfort, etc.

The present invention thus relates to an extract of bacterium of Sphingomonas xenophaga species.

Another object of the present invention is a composition comprising at least one bacterium of Sphingomonas xenophaga species or an extract of bacterium of Sphingomonas xenophaga species. The composition according to the invention is preferably cosmetic.

The present invention also relates to the use of at least one bacterium of Sphingomonas genus or an extract of bacteria of Sphingomonas genus as an inhibitor of bradykinin synthesis.

Another object of the present invention is a non-therapeutic cosmetic treatment method for redness of the skin, comprising the topical application of at least one bacterium of Sphingomonas genus or an extract of bacteria of Sphingomonas genus, or a composition comprising at least one bacterium of Sphingomonas genus or an extract of bacteria of Sphingomonas genus.

The present invention also relates to a non-therapeutic cosmetic treatment method for the reactive skin, comprising the topical application of at least one bacterium of Sphingomonas genus or an extract of bacteria of Sphingomonas genus, or a composition comprising at least one bacterium of Sphingomonas genus, or an extract of bacteria of Sphingomonas genus.

### DETAILED DESCRIPTION OF THE INVENTION

### Extract of Bacteria of Sphingomonas Genus

The term “bacterium” designates a prokaryotic microorganism, preferably a proteobacterium.

In the framework of the invention, the term “extract of bacteria” designates both a set of compounds produced and/or secreted by a bacterium, therefore present in the culture medium of the bacterial culture, and/or a set of compounds comprised in the bacterium, therefore present in the bacterial pellet of the bacterial culture after centrifugation, such as the intracellular medium and/or the constituents of the walls and/or the cell membranes.

The extracts according to the invention can be prepared by any conventional method well known to persons skilled in the art. These methods typically comprise a step of fermentation, isolation, separation and purification.

Typically, the bacterium of Sphingomonas genus is cultivated in a suitable medium and cultivated at a desired density. Preferably, the bacteria themselves are concentrated by any known method, such as centrifugation. The concentrated bacteria can then be used directly as a raw preparation, or additional steps of treatment well known to person skilled in the art can be carried out, such as lyophilization, dehydration, filtration, purification, freezing optionally followed by a thawing, sterilization, column chromatography, grinding, etc.

Preferably, the bacterial extract is a lysate of bacteria comprising in particular the compounds comprised in the bacterium, therefore present in the bacterial pellet of the bacterial culture after centrifugation. Preferably, the bacterial extract is a lysate of bacteria comprising the intracellular medium and/or the constituents of the walls and/or cell membranes.

A lysate commonly designates a material obtained at the end of the destruction or dissolution of biological cells by a phenomenon called cell lysis thus causing the release of the intracellular biological constituents naturally contained in the cells of the microorganism considered.

In terms of the present invention, the term lysate is used indifferently to designate all the lysate obtained by lysis of the microorganism concerned or only a fraction of the latter.

The lysate implemented is therefore form entirely or partially of the intracellular biological constituents and constituents of the cell walls and membranes.

This cellular lysis can be carried out using different technologies, such as for example osmotic shock, thermal shock such as a freezing optionally followed by a thawing, by ultrasound, or under mechanical stress of the centrifugation type for example.

Preferably, the lysate implemented in the framework of the present invention is obtained by a method comprising the following steps:
i) a step of centrifugation;
ii) a step of recovering the pellet;
iii) a step of freezing the pellet;
iv) a step of thawing the pellet;
v) optionally a step of sterilizing via autoclaving, in particular at 121° C.

In a particular embodiment of the invention the extract of bacteria comprises an inactivated bacterium.

Here, “inactivated” has the meaning that is commonly used by persons skilled in the art, i.e. the suppression of the activity of the bacterium under the effect of various causes (heat, chemical, mechanical, enzyme substances). In particular, the bacterium is inactivated by heat, more particularly by autoclaving.

Said extract preferably has an inhibiting activity of bradykinin.

The bacterium of the extract according to the invention is Sphingomonas xenophaga. In a preferred embodiment the bacterium is a Sphingomonas xenophaga registered according to the Budapest Treaty, on Nov. 21, 2019, with the Collection Nationale de Culture de Microorganismes ((CNCM), Paris, France) under the number CNCM I-5455 by L'Oréal, 101 Avenue Gustave Eiffel, 37390 Notre Dame d'Oé.

Preferably, the extract is contained in a cosmetic composition in a cosmetically acceptable medium.

### Composition

The present invention also relates to a composition, in particular a cosmetic composition, comprising at least one bacterium of Sphingomonas xenophaga species or an extract as defined hereinabove.

Preferably, the cosmetic composition according to the invention is intended to be administered topically.

Here, “cosmetic composition” has the meaning commonly used by persons skilled in the art. In particular, it means a substance or a preparation intended to be placed in contact with the various surface portions of the human body, in particular the epidermis, hair systems, nails, lips and external genital organs, and the mucous membranes of the oral cavity, with a view exclusively or mainly to cleaning them, embellishing them, perfuming them, modifying their aspect, protecting them, maintaining them in good condition or correcting body odors.

“Bacterium of Sphingomonas xenophaga species” means a bacterium in a live, semi-active, inactivated or dead form.

The term “extract of bacteria” is as defined in the paragraph Extract of bacteria of Sphingomonas genus.

The quantity of bacteria or extract of bacteria in the composition according to the invention varies according to the type of composition considered. Preferably, the quantity of microorganism or extract is comprised between 0.0001 and 30% by dry weight in relation to the total weight of the composition, between 0.001 and 15% by dry weight in relation to the total weight of the composition, between 0.01 and 10% by dry weight in relation to the total weight of the composition, preferably between 0.01 and 5% by dry weight in relation to the total weight of the composition, or between 0.01 and 3% by dry weight in relation to the total weight of the composition.

In the case where the bacteria are formulated in a composition in a live form, the quantity of living bacteria can vary from 10³ to 10¹⁵ cfu/g, in particular from 10⁵ to 10¹⁵ cfu/g and more particularly from 10⁷ to 10¹² cfu/g of bacteria per gram of composition.

Preferably, the composition comprises a cosmetically acceptable medium.

Here, “cosmetically acceptable medium” means a non-toxic support that can be applied on keratin materials such as the skin and mucous membranes. Thus a cosmetically acceptable medium is compatible with the skin, teguments and/or mucous membranes, it does not induce any sensations of discomfort, or more generally disorders able to lead the user to suspend or stop the administration of the cosmetic composition.

More particularly, said cosmetically acceptable medium can comprise water and/or one or more water-miscible organic solvents that can be selected from straight or branched C1-C6 mono-alcohols such as ethanol, isopropanol, tertio-butanol; polyols such as glycerol, propyleneglycol, hexylene glycol (o 2-methyl-2,4-pentanediol), and polyethyleneglycols; polyol ethers such as dipropyleneglycol monomethylether; and mixtures thereof.

Preferably, the composition according to the invention has a water content ranging from 20% to 95% by weight, more preferably from 30% to 70% by weight in relation to the total weight of the composition.

Advantageously, the composition comprises one or more water-miscible organic solvents in a content ranging from 0.5% to 25% by weight, preferably, from 5% to 20% by weight, more preferably from 10% to 15% by weight in relation to the total weight of the composition.

As is well known to a person skilled in the art, compositions for topical application can also contain additives common in the cosmetics field, such as hydrophilic or lipophilic gelling agents, hydrophilic or lipophilic active agents different from the extract according to the invention, preservatives, antioxidants, scents, odor-absorbing agents, colorants, and mixtures thereof.

Preferably, said composition further comprises:
at least one additives chosen from hydrophilic or lipophilic gelling agents, hydrophilic or lipophilic active agents different from the extract according to the invention, preservatives, antioxidants, scents, odor-absorbing agents, colorants, and mixtures thereof; and/or
at least one emulsifier; and/or
at least one oil.

The quantities of these various additives are conventionally the quantities used in the particular domain, for example from 0.01% to 20% of the total weight of the composition.

Obviously, those skilled in the art shall choose these additional ingredients and/or active agents, and/or the quantity thereof, such that the advantageous properties of the extract according to the invention are not, or are substantially not, altered by the contemplated addition.

In an embodiment, the bacteria of Sphingomonas genus or the extract of bacteria of Sphingomonas genus is the only active agent of the composition.

In another embodiment, the composition of the invention can furthermore contain other active agents different from the bacteria or the extract according to the invention. These active agents can have an effect on the vasodilation, redness, reactive skins or on other dysesthetic elements.

In a particular embodiment of the invention, the composition according to the invention further comprises at least one active agent selected from acetyl dipeptide-1 cetyl ester, an extract of the root of Salvia miltiorrhiza, a spring water such as La Roche Posay, and mixtures thereof.

The compositions according to the invention can have all the dosage forms conventionally used for a topical application and in particular in the form of aqueous solutions, hydroalcoholic solutions, oil-in-water (O/W) or water-in-oil (W/O) or multiple (triple: W/O/W or O/W/O) emulsions, aqueous gels, or dispersions of an oily phase in an aqueous phase using spheres, these spheres potentially being lipid vesicles of the ionic and/or non-ionic type (liposomes, niosomes, oleosomes). These compositions are prepared using routine methods.

Advantageously the compositions according to the invention have the form of a gel, or an emulsion, powder or paste. Furthermore, the composition according to the invention can be more or less fluid and have the appearance of a white or colored cream, an ointment, a milk, a lotion, a serum, a paste, a foaming gel, a peeling, a mask, a care, a tonic or a foam. It can optionally be applied on the skin in the form of an aerosol. It can also have a solid form, and for example have the form of a stick.

A composition according to the invention may comprise an oily phase.

As oils suitable for use in the composition according to the invention, mention may be made for example of:
hydrocarbon oils,
synthetic esters and ethers, in particular of fatty acids, such as the oils of formulas R′COOR2 and R′OR2 wherein R′ represents the residue of a fatty acid including 8 to 29 carbon atoms, and R2 represents an optionally branched hydrocarbon chain, containing 3 to 30 carbon atoms;
linear or branched hydrocarbons of mineral or synthetic origin;
fatty alcohols having from 8 to 26 carbon atoms;
partially hydrocarbon and/or silicone fluorinated oils;
silicone oils;
mixtures thereof.

The term “hydrocarbon oil” in the list of oils cited above means any oil including mostly carbon and hydrogen atoms.

The oily phase can contain other fatty bodies that can be present in the oily phase are for example fatty acids including 8 to 30 carbon atoms; waxes; silicone resins; and silicone elastomers. These fats may be chosen in varied ways by those skilled in the art in order to prepare a composition having the desired properties, for example in terms of consistency or texture.

According to a particular embodiment of the invention, the composition according to the invention is a water-in-oil (W/O) or oil-in-water (O/W) emulsion. The proportion of the oily phase of the emulsion may range from 5 to 80% by weight, and preferably from 5 to 60% by weight relative to the total weight of the composition. The emulsions generally contain at least one emulsifier chosen from amphoteric, anionic, cationic or non-ionic emulsifiers, used alone or in a mixture, and optionally a co-emulsifier. The emulsifiers are appropriately selected according to the emulsion to be obtained (W/O or O/W). The emulsifier and the co-emulsifier co-emulsifier are generally present in the composition, in a proportion ranging from 0.3% to 30% by weight, and preferably from 0.5% to 20% by weight, relative to the total weight of the composition.

For W/O emulsions, as emulsifiers mention can be made for example of dimethicone copolyols, and alkyl-dimethicone copolyols. It is also possible to use as a surfactant of W/O emulsions, a cross-linked solid elastomeric organopolysiloxane including at least one oxyalkylene group.

For O/W emulsions, as emulsifiers mention can be made for example of non-ionic emulsifiers.

The compositions according to the invention can be applied directly on the skin or, alternatively, on cosmetic supports of the occlusive or non-occlusive type, intended to be applied locally on the skin. As non-limiting examples of cosmetic supports, mention can be made of a patch, a towelette, a mask on a support. The composition can be rinsed or not after having been applied to the skin.

In a preferred embodiment, the compositions used in the framework of the invention are intended for topical administration. In a preferred embodiment, the compositions used in the framework of the invention are intended for application on the skin.

In the framework of the invention, the term “skin” designates any cutaneous surface of the body, preferably the skin of the face or the legs, and more particularly the skin of the face.

### Uses

The invention relates to the use of at least one bacterium of the Sphingomonas genus or an extract of bacteria of Sphingomonas genus as an inhibitor of bradykinin.

The bacterium used according to the invention or the bacterium of the extract used according to the invention is of Sphingomonas genus. In an embodiment, the bacterium is of a species selected from Sphingomonas abaci, Sphingomonas adhaesiva, Sphingomonas aerolata, Sphingomonas aquatilis, Sphingomonas asaccharolytica, Sphingomonas aurantiaca, Sphingomonas azotifigens, Sphingomonas dokdonensis, Sphingomonas echinoides, Sphingomonas faeni, Sphingomonas fennica, Sphingomonas haloaromaticamans, Sphingomonas jaspsi, Sphingomonas koreensis, Sphingomonas mall, Sphingomonas melonis, Sphingomonas natatoria, Sphingomonas oligophenolica, Sphingomonas panni, Sphingomonas parapaucimobilis, Sphingomonas paucimobilis, Sphingomonas phyllosphaerae, Sphingomonas pituitosa, Sphingomonas pruni, Sphingomonas roseiflava, Sphingomonas sanguinis, Sphingomonas soli, Sphingomonas sp, Sphingomonas suberifaciens, Sphingomonas trueperi, Sphingomonas Sphingomonas xenophaga, Sphingomonas yabuuchiae, and Sphingomonas yunnanensis, Sphingomonas yanoikuyae. In an embodiment of the invention, the bacterium is Sphingomonas xenophaga. In a preferred embodiment the bacterium is a Sphingomonas xenophaga registered according to the Budapest Treaty, on Nov. 21, 2019, with the Collection Nationale de Culture de Microorganismes ((CNCM), Paris, France) under the number CNCM I-5455 by L'Oréal, 101 Avenue Gustave Eiffel, 37390 Notre Dame d'Oé.

Preferably, the uses according to the invention relate to the use of at least one bacterium of Sphingomonas genus or an extract of bacteria of Sphingomonas genus or a preferably cosmetic composition according to the invention.

Preferably, the use according to the invention is a topical use. Preferably, the bacterium or the extract of bacterium or the composition containing it is applied on the skin, more preferably on the face, the legs or the body, more preferably on the face.

The term “inhibitor of bradykinin” means a compound that has for effect to inhibit or reduce the biological activity of bradykinin. In an embodiment the extract of bacteria of Sphingomonas genus of the invention inhibits a reaction upstream of the activation of the bradykinin, for example by inhibiting or reducing the activation of the kallikrein/kinin system (KKS), or by inhibiting or reducing the activation of the prekallikrein. Preferably, the extract of bacteria of Sphingomonas genus is used as an inhibitor of the activation of prekallikrein.

This inhibition results in the inhibition of the vasodilation and the increase in the vascular permeability.

Thus the invention also relates to the use of at least one bacterium of Sphingomonas genus or an extract of bacteria of Sphingomonas genus or a composition containing it as an inhibitor of vasodilation and/or increase in vascular permeability.

The invention concerns the use of at least one bacterium of Sphingomonas genus or an extract of bacteria of Sphingomonas genus or a composition containing it for the prevention and/or the treatment of cosmetic disorders associated with an excess in synthesis and/or release of bradykinin.

Here, the term “prevent” or “prevention” designates any action that aims to prevent the appearance of a discomfort or uneasiness of an individual. This terms therefore covers the slowing down, stoppage or limitation of the symptoms, i.e. of dysesthetic sensations, but is limited to the cosmetic aspects.

Here, the term “treat” or “treatment” designates any action that aims to improve the comfort or the well-being of an individual. This terms therefore covers the attenuation, easing or suppression of the symptoms, i.e. of dysesthetic sensations, but is limited to a cosmetic treatment.

The term “cosmetic disorders” means signs of discomfort, dysesthetic sensations, such as for example more or less unpleasant sensations felt on a skin zone, such as tautness, tingling, itching, heating. It also means visible manifestations such as redness, cutaneous vasodilations and/or telangiectasia.

The term “excess in synthesis and/or release of bradykinin” means a high synthesis of bradykinin and/or a high release of bradykinin on the skin of the subject with respect to the average level of synthesis and/or release observed in this subject. The term “high” means here an increase of at least 20%, at least 50% or at least 100%.

Preferably, the use of at least one bacterium of Sphingomonas genus or an extract of bacteria of Sphingomonas genus or a composition containing it according to the invention is for preventing and/or decreasing the redness, cutaneous vasodilations and/or telangiectasia.

Here, the term “redness” or “red patch” designates a pink to red, even dark red, coloration of all or a portion of the skin of the body, scalp, mucous membranes or semi-mucous membranes. This manifestation, also called erythema, can be the sign of a healthy complexion (rosy cheeks), but, although benign, it is however sometimes undesirable and considered as unsightly.

This redness can be triggered by a great variety of factors, such as (i) external stimuli such as the consumption of food or hot or alcoholic beverages, by rapid variations in temperature, by heat or cold, by a low relative humidity, by exposure of the skin to violent winds or drafts (fans, air conditioning), and by the application of certain surfactants, or (ii) redness associated with a skin disorder such as rosacea, acne rosacea, atopic dermatitis, seborrhoeic dermatitis, as well as (iii) redness subsequent to an allergic skin reaction.

The term “external stimulus” means a stimulus that is not able itself to cause skin lesions, preferably selected from heat, cold, a low relative humidity, exposure of the skin to violent wind or to drafts.

Here, the term “cutaneous vasodilations” designates a benign vasodilation on all or a portion of the skin of the body, the scalp, mucous membranes or semi-mucous membranes, it is sometimes undesirable and considered as unsightly. This type of vasodilations is often temporary.

Preferably, the use of at least one bacterium of Sphingomonas genus or an extract of bacteria of Sphingomonas genus or a composition containing it according to the invention is for preventing and/or decreasing cutaneous vasodilations induced by bradykinin.

Here, the term “telangiectasia” designates a permanent abnormal dilation of the small blood vessels located near the surface of the skin, of mucous membranes. They are typically of red to violet color with a thin line and winding as a tree or network. They appear more frequently on the face or legs.

The invention also relates to the use of at least one bacterium of Sphingomonas genus or an extract of bacteria of Sphingomonas genus or a composition containing it according to the invention is for preventing and/or decreasing skin discomfort characterized by tautness, heating and/or itching.

### Methods

The invention relates to a non-therapeutic cosmetic treatment method for redness of the skin, comprising the topical application of at least one bacterium of Sphingomonas genus or an extract of bacteria of Sphingomonas genus or a composition comprising it.

Preferably, the application is carried out on a subject that needs it and in an effective quantity of at least one bacterium of Sphingomonas genus or at least one extract of bacteria of Sphingomonas genus.

Here, the term “effective quantity” refers to the quantity of extract of bacteria of Sphingomonas genus, that, as a whole, makes it possible to prevent and/or decrease redness of the skin or the quantity of bacteria of Sphingomonas genus, that, as a whole, makes it possible to prevent and/or decrease redness of the skin.

In the framework of the invention, the term “skin” designates any cutaneous surface of the body, preferably the skin of the face.

The invention also relates to a non-therapeutic cosmetic treatment method for the reactive skin, comprising the topical application of at least one bacterium of Sphingomonas genus or an extract of bacteria of Sphingomonas genus or a composition comprising it.

Here, the term “reactive skin” designates an intolerant skin that reacts through sensations of heating, tautness and/or tingling, to various factors such as a cold or hot application.

In a particular embodiment, the cosmetic treatment method for reactive skin according to the invention aims to prevent and/or attenuate the sensations of heating, tautness and/or tingling of the skin.

Preferably, the application is carried out on a subject that needs it and in an effective quantity of at least one bacterium of Sphingomonas genus or at least one extract of bacteria of Sphingomonas genus.

Here, the term “effective quantity” refers to the quantity of extract of bacteria of Sphingomonas genus, that, as a whole, makes it possible to treat the reactive skin or to the quantity of bacteria of Sphingomonas genus, that, as a whole, makes it possible to treat the reactive skin.

The bacterium applied or bacterium of the extract applied in the methods according to the invention is of Sphingomonas genus. In an embodiment, the bacterium is of a species selected from Sphingomonas abaci, Sphingomonas adhaesiva, Sphingomonas aerolata, Sphingomonas aquatilis, Sphingomonas asaccharolytica, Sphingomonas aurantiaca, Sphingomonas azotifigens, Sphingomonas dokdonensis, Sphingomonas echinoides, Sphingomonas faeni, Sphingomonas fennica, Sphingomonas haloaromaticamans, Sphingomonas jaspsi, Sphingomonas koreensis, Sphingomonas mall, Sphingomonas melonis, Sphingomonas natatoria, Sphingomonas oligophenolica, Sphingomonas panni, Sphingomonas parapaucimobilis, Sphingomonas paucimobilis, Sphingomonas phyllosphaerae, Sphingomonas pituitosa, Sphingomonas pruni, Sphingomonas roseiflava, Sphingomonas sanguinis, Sphingomonas soli, Sphingomonas sp, Sphingomonas suberifaciens, Sphingomonas trueperi, Sphingomonas wiffichii, Sphingomonas xenophaga, Sphingomonas yabuuchiae, and Sphingomonas yunnanensis, Sphingomonas yanoikuyae. In a particular embodiment the bacterium is Sphingomonas xenophaga. In a preferred embodiment the bacterium is a Sphingomonas xenophaga registered according to the Budapest Treaty, on Nov. 21, 2019, with the Collection Nationale de Culture de Microorganismes ((CNCM), Paris, France) under the number CNCM I-5455 by L'Oréal, 101 Avenue Gustave Eiffel, 37390 Notre Dame d'Oé.

Preferably, the methods according to the invention comprise the topical application of at least one bacterium of Sphingomonas genus or at least one extract of Sphingomonas genus or a preferably cosmetic composition comprising it, according to the invention. Preferably, the bacterium, the extract or the composition is applied on the face, legs or the body, more preferably the face.

Here, the term “subject” means a human being.

According to the present invention, the methods are non-therapeutic methods.

The methods of the invention can comprise a single administration. In another embodiment, the administration is repeated for example 2 to 3 times per day for one day or more and generally during an extended period of at least 4 weeks or 4 to 15 weeks, or as long as necessary with a possible break if needed.

In the description and in the examples hereinafter, unless specified otherwise, the percentages are percentages by weight and the value ranges worded as “between . . . and . . . ” comprise the upper and lower limits specified. The ingredients are mixed, prior to the packaging thereof, in order and under conditions easily determined by those skilled in the art.

The present invention will furthermore be illustrated by the examples hereinbelow.

### FIGURES

FIG. 1 shows the Vmax of the plasma prekallikrein according to the concentration as a percentage of the extract of Sphingomonas.

FIG. 2 is a histogram showing the percentage of activity the plasma prekallikrein according to the concentration the extract of Sphingomonas used.

FIG. 3 is a histogram that shows the change in the clinical score of the redness over time.

FIG. 4 is a histogram that shows the change in the score by self-evaluation of the redness over time.

### EXAMPLES

### Example 1: Preparation of an Extract According to the Invention

A culture of the Sphingomonas xenophaga CNCM-I 5455 strain is carried out in its complete culture medium in a bioreactor of 3000 effective liters in batch mode. During this step, the pH is not adjusted, the temperature is maintained at 26° C. and the oxygen dissolved at 30%.

The composition of the initial culture medium is described in Table 1 hereinbelow.

TABLE 1

| Chemical name | concentration | |
|---|---|---|
| Autolytic extract of yeast | 4 | g/l |
| Papainic peptone of soja F | 3 | g/l |
| Glucose | 3 | g/l |
| KH₂PO₄ | 0.088 | g/l |
| CaCl₂ | 0.050 | g/l |
| CuSO₄, 5 H₂O | 60 | μg/l |
| MnSO₄, 1H₂O | 152 | μg/l |
| KI | 20 | μg/l |
| ZnSO₄, 7H₂O | 200 | μg/l |
| AlCl₃, 6H₂O | 100 | μg/l |

| | |
|---|---|
| Reverse osmosis water | qs 1 l |

As soon as the plateau phase is reached, the extraction and the separation of the cells are carried out by centrifugation (10,000 g continuously). Then, the pellet also called biomass containing the cells is recovered, to then be frozen at −20° C. then thawed allowing for the bursting of the cells and thus the obtention of a lysate. The lysate is then conditioned in bags and finally stabilized by sterilization at 121° C. for 30 minutes.

The lysate obtained at the end of the method as described according to example 1 contains 4.5% by weight of dry matter, with respect to the total weight of the lysate.

### Example 2: In Vitro Test—a Dose/Response Curve of the Effect of the Extract of Sphingomonas on the Activation of Prekallikrein of a Normal Plasma

The effect of the extract of example 1 on the substrate is monitored by the measurement by spectrophotometry (λ=405 nm) of the cleavage of a chromogenic substrate.

The enzymatic activities are evaluated by the monitoring of the kinetic reaction and the calculation of the maximum speed of the reaction. The results represent the average plus or minus the standard deviation of at least two duplicates made on two independent experiments. The statistical tests are conducted with the Mann Whitney test α<0.05.

The extract has a constant but intrinsic absorption at the wavelength of 405 nm, it was therefore necessary for all of the experiments to subtract this specific absorption from the absorptions measured.

The activation of the prekallikrein is evaluated after 10 min of incubation of a normal plasma with the extract tested at 0° C. at the concentrations of 0.02; 0.1; 0.2; 0.3; 0.4 and 0.5%, followed by a complete activation of the prekallikrein thanks to the dextran sulfate.

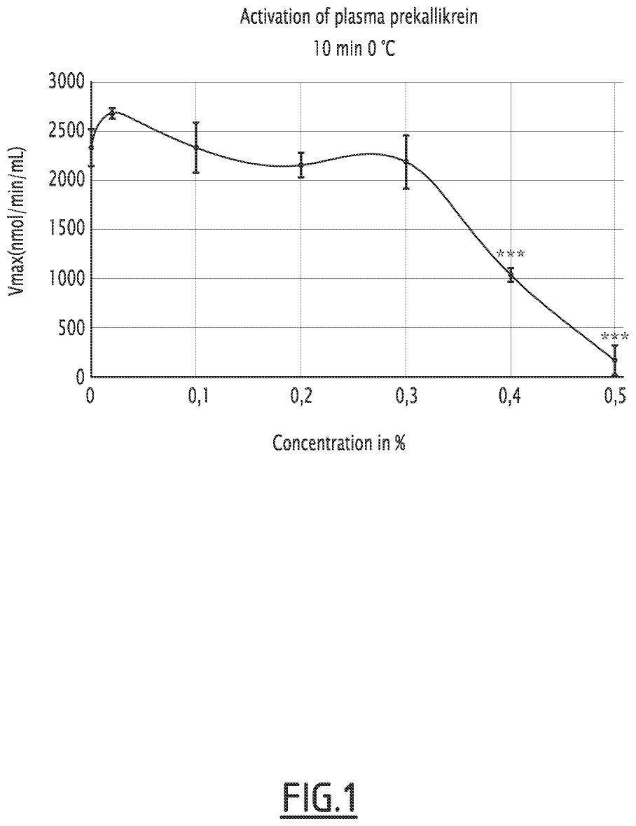

FIG. 1 shows the impact of the extract of Sphingomonas on the activation of prekallikrein in the plasma.

At the concentrations of 0.02; 0.1; 0.2 and 0.3% the extract does not show any significant inhibitory effect (p>0.05). The inhibitory effect can be observed starting at the concentration of 0.4%. The inhibition of the kallikrein-kinin system is 46±8 and 90±8% at the final concentrations of 0.4 and 0.5% (p<0.001; FIG. 1). The inventors have thus confirmed the capacity of the extract to inhibit the activation of the kallikrein-kinin system.

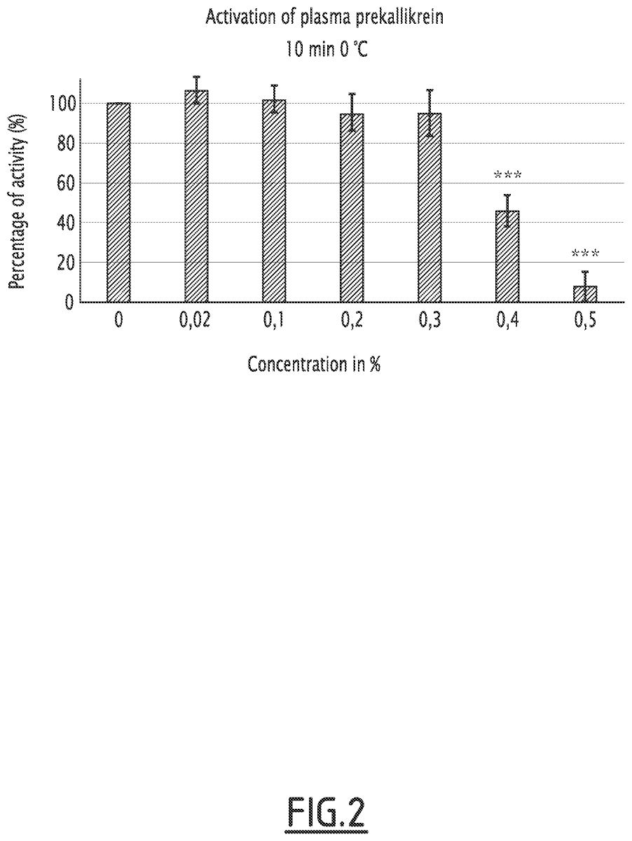

This can also be observed in FIG. 2 which shows the percentage of the activity of the plasma prekallikrein according to the concentration of the extract used.

In conclusion, the data obtained with the extract of Sphingomonas on the kallikrein-kinin system confirm the inhibitory effect on the activation of prekallikrein in a normal plasma in the conditions of the experience (10 min of incubation at 0° C. before activation of the plasma). In these conditions, the inhibitory effect is significant starting from the concentration of 0.4% of the extract of example 1.

### Example 3: In Vivo Test—Evaluation of the Soothing Effect of the Extract of Sphingomonas

This test is conducted on 10 persons distributed into two groups. It consists of the application of capsaicin at a concentration of 3.16×10⁻³% on a zone of the skin of the face of the subjects of the first group, followed by the topical application of a composition comprising the extract of Sphingomonas of example 1. The second group is a control group that receives the treatment with capsaicin but not the composition comprising the extract of Sphingomonas. The skin stimulated by the capsaicin is here used as a reactive skin model.

The composition comprising the extract of Sphingomonas used a for composition:

TABLE 2

| | % Concen- |
|---|---|
| Chemical Name | tration |
| MICROBIOLOGICALLY CLEAN DEIONIZED WATER | qs 100 |
| 1,3-BUTYLENE GLYCOL | 5 |
| PRESERVATIVES | qs |
| ETHYLENEDIAMINE DISUCCUNIC ACID, | 0.2 |
| TRISODIUM SALT IN AQUEOUS SOLUTION AT 30% | |
| GLYCERIN | 7 |
| N-ACETYL-TYR-ARG-HEXADECYL ESTER WITH | 0.0337 |
| 5% HYDROCHLORIC ACID | |
| MONO-LAURATE OF SORBITANE OXYETHYLENE | 1 |
| (20 EO) | |
| CITRIC ACID, 1 H₂O | 0.0285 |
| POLYACRYLAMIDOMETHYL PROPANE SULFONIC | 0.9 |
| ACID PARTIALLY NEUTRALIZED WITH AMMONIA | |
| AND HIGHLY CROSS-LINKED | |
| 2,6,10,15,19,23-HEXAMETHYL- | 2 |
| DODECAHYDROSQUALENE | |
| ACTIVE AGENT | 3 |
| ACRYLIC POLYMER IN WATER/GLYCERINE AT 1% | 5 |
| WITHOUT PRESERVATIVE | |
| CORN STARCH | 0.75 |
| ROCHE-POSAY WATER | 5 |
| EXTRACT OF SPHINGOMONAS FERMENT WITH | 0.2 |
| WATER | |
| BUTYLENE GLYCOL AND WATER AND EXTRACT | 2.5 |
| OF THE ROOT OF SALVIA MILTIORRHIZA | |

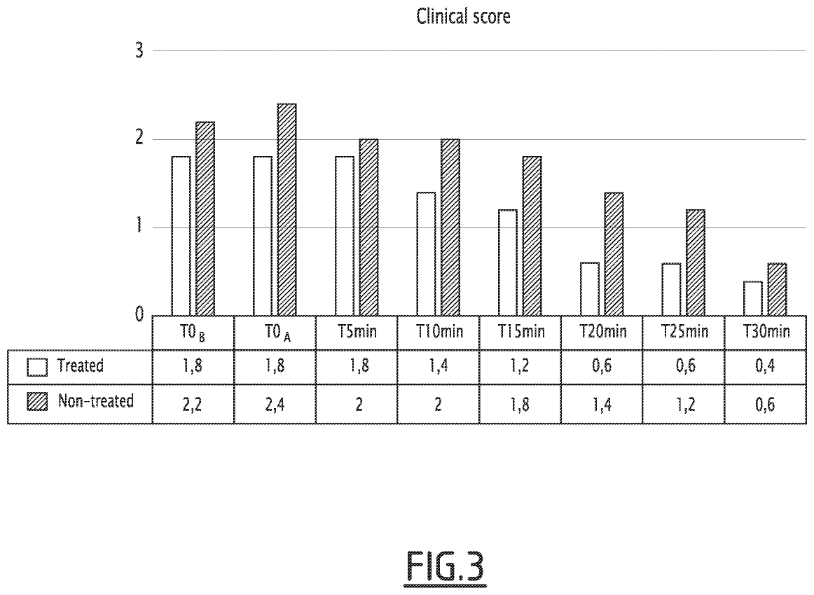

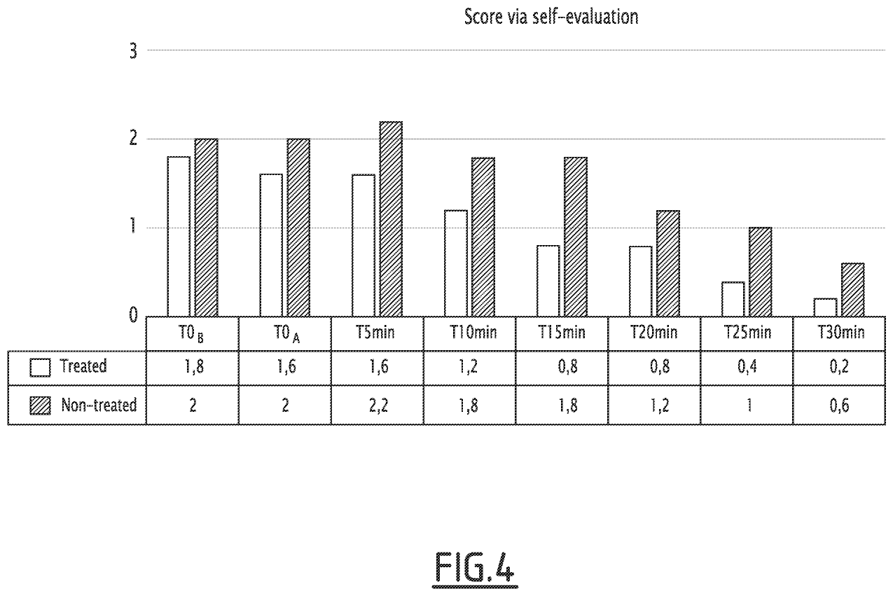

The redness of the skin on the zone tested is rated from 0 to 4 by a clinician or the subject themselves. Thus the level of the redness is rated:
after application of the capsaicin and before application of the composition (T0_{B}),
immediately after the application of the composition (T0_{A}), then
at 5, 10, 15, 20, 25 and 30 minutes after the application of the capsaicin.
   The change in the clinical score is shown in FIG. 3. The change in the score given by the subjects themselves (therefore a self-evaluation) is shown in FIG. 4. It is observed that on the two graphs the score is lower for the group of people who received the composition comprising the extract of Sphingomonas than for the control group. In these conditions, the composition comprising the extract of Sphingomonas made it possible to decrease redness.

1. An extract of bacteria of Sphingomonas xenophaga species.

2. The extract according to claim 1, wherein the strain of Sphingomonas xenophaga is the strain registered under accession number CNCM I-5455.

3. A composition, comprising an extract according to claim 1 or at least one bacterium of Sphingomonas xenophaga species as described therein.

4. The composition according to claim 3, wherein the concentration of the extract is between 0.0001% and 30% by weight with respect to the total weight of the composition.

5. The composition according to claim 3, further comprising at least one active agent selected from the group consisting of acetyl dipeptide-1 cetyl ester, an extract of the root of Salvia miltiorrhiza, a spring water and mixtures thereof.

6. A composition according to claim 3, wherein said composition comprises a cosmetically acceptable medium comprising one or more water-miscible organic solvents.

7. A composition according to claim 3 wherein said composition further comprises:
a. at least one additive chosen from hydrophilic or lipophilic gelling agents, hydrophilic or lipophilic active agents different from the extract according to the invention, preservatives, antioxidants, scents, odor-absorbing agents, colorants, and mixtures thereof; and/or
b. at least one emulsifier; and/or
c. at least one oil.

8. A method for inhibiting an in excess in synthesis and/or release of bradykinin in a subject in need thereof with comprises administering to the subject at least one bacterium of Sphingomonas genus or an extract of bacteria of Sphingomonas genus as an inhibitor of bradykinin.

9. The method according to claim 8 comprising the prevention and/or the treatment of cosmetic disorders associated with an excess in the synthesis and/or release of bradykinin.

10. The method according to claim 8 comprising preventing and/or decreasing the redness, cutaneous vasodilations and/or telangiectasia.

11. The method according to claim 8 comprising preventing and/or decreasing skin discomfort characterized by tautness, tingling, heating and/or itching.

12. A non-therapeutic cosmetic treatment method for the redness of the skin, comprising the topical application of at least one bacterium of Sphingomonas genus or at least one extract of bacteria of Sphingomonas genus, or a composition comprising at least one bacterium of Sphingomonas genus or an extract of bacteria of Sphingomonas genus.

13. A non-therapeutic cosmetic treatment method for the reactive skin, comprising the topical application of at least one bacterium of Sphingomonas genus or at least one extract of Sphingomonas genus, or a composition comprising at least one bacterium of Sphingomonas genus or an extract of bacteria of Sphingomonas genus.

14. A composition comprising an extract according to claim 2 or at least one bacterium of Sphingomonas xenophaga species as described therein.

15. The composition according to claim 4, further comprising at least one active agent selected from the group consisting of acetyl dipeptide-1 cetyl ester, an extract of the root of Salvia miltiorrhiza, a spring water and mixtures thereof.

16. A composition according to claim 4, wherein said composition comprises a cosmetically acceptable medium comprising one or more water-miscible organic solvents.

17. A composition according to claim 5, wherein said composition comprises a cosmetically acceptable medium comprising one or more water-miscible organic solvents.

18. A composition according to claim 4 wherein said composition further comprises:
a. at least one additive chosen from hydrophilic or lipophilic gelling agents, hydrophilic or lipophilic active agents different from the extract according to the invention, preservatives, antioxidants, scents, odor-absorbing agents, colorants, and mixtures thereof; and/or
b. at least one emulsifier; and/or
c. at least one oil.

19. A composition according to claim 5 wherein said composition further comprises:
d. at least one additive chosen from hydrophilic or lipophilic gelling agents, hydrophilic or lipophilic active agents different from the extract according to the invention, preservatives, antioxidants, scents, odor-absorbing agents, colorants, and mixtures thereof; and/or
e. at least one emulsifier; and/or
f. at least one oil.

20. A composition according to claim 6 wherein said composition further comprises:
g. at least one additive chosen from hydrophilic or lipophilic gelling agents, hydrophilic or lipophilic active agents different from the extract according to the invention, preservatives, antioxidants, scents, odor-absorbing agents, colorants, and mixtures thereof; and/or
h. at least one emulsifier; and/or
i. at least one oil.
